(19) United States
(12) Patent Application Publication
(10) Pub. No.: US 2019/0054308 A1
(43) Pub. Date: Feb. 21, 2019

(54) Pulsed Electromagnetic Field (PEMF) Therapy Whole Body Wellness Device to increase cells energy, strengthen immune system and promote cell regeneration

(71) Applicant: Sandhya Verma, Brampton (CA)

(72) Inventor: Sandhya Verma, Brampton (CA)

(21) Appl. No.: 15/917,011

(22) Filed: Mar. 9, 2018

Related U.S. Application Data
(60) Provisional application No. 62/547,293, filed on Aug. 18, 2017.

(51) Int. Cl. A61N 2/00; A61N 2/02

(52) U.S. Cl. CPC A61N 2/008; A61N 2/002; A61N 1/40; A61N 2/006; A61N 2/02

(57) ABSTRACT

Systems and techniques for applying a pulsed electromagnetic field to body include: Self-contained portable pulsed electromagnetic field generating device which creates electromagnetic pulses given to feet and hands of the body. This device includes an electromagnetic field generator to provide power, device base to receive pulses from feet and device bulb to receive pulses from hand, thereby increasing energy of cells in the body.

In PEMF-DS100 Therapy, the person's body is placed through the electromagnetic field to deliver treatment. Various specialized materials (described in detail below) are used to produce an electromagnetic field or Induction, which is given or passed through the whole body via both hands and feet.

### Aluminum Base:

### PEMF and MWO Pulses or Induction Given Through Feet (Aluminum Base)

1) The base material used is lexan or polycarbonate or plastic material ½ inch thickness (size 24 inches by 24 inches)
2) Then clear known standard glass is placed on top of lexan (size 24 inches by 24 inches)
3) Then standard Aluminum known in the field is placed on top of clear glass (size 18 inches by 18 inches) with a notch of 4 inches for connection
4) Then clear known standard black glass known in the field placed on top of Aluminum (size 24 inches by 24 inches)
5) Join all these pieces from all four sides with 3 mm acrylic
6) Have a electrical connection from Aluminum going out via % inch standard audio jack—male end (connection 1 to Electrical Unit)

### Coil or Copper House

### PEMF and MWO Pulses or Induction Given Through Coil or Copper House

1) The material used in coil or copper house is acrylic 5 mm and make a house shape with acrylic size 8 inches by 10 inches at the base and 5 inches high
2) At the base put a piece of standard coil or copper known in the field
3) On top put a standard fan known in the field in the middle round circle
4) Have a electrical connection from coil or copper going out via ¼ inch standard audio jack—male end (connection 2 to Electrical Unit)
5) On top at the round circle—Fan is installed
6) At bottom—coil or copper is installed
7) Place this coil or copper house on top of the base which is explained above.

### PEMF and MWO Pulses or Induction Given Through Hand (Bulb)

1) Also on the side have a standard bulb known in the field size with electrical connection going out via ¼ inch standard audio jack—male end and place it on the side of the base. (connection 3 to Electrical Unit)

### Standard Known Electrical Unit

1) Attach Connection 1, 2 and 3 to Electrical Unit via ¼ inch standard audio jack—female end to send PEMF and MWO pulses or induction throughout the whole body
2) Use standard Electrical Unit known in the field to give power
3) Attach 3 connections to the Electrical Unit

### Method of USE (Instructions on How to Use)

1) Switch the Electrical Unit on
2) Sit comfortably on plastic or wooden chair
3) Place the Bulb in both the hands
4) Then place both the feet on the base
5) Let the electromagnetic field or Induction pass through the hands and feet of the body for half an hour to less than an hour depending upon the condition (3 to 4 times per day)

### BRIEF DESCRIPTION OF THE SEVERAL VIEWS OF THE DRAWING

Embodiments are illustrated by way of examples in the drawings which have been faxed on 18 May, 2018, in which:

FIG. 1 provides a perspective view of embodiment of Aluminum Base from where the induction is given to the feet

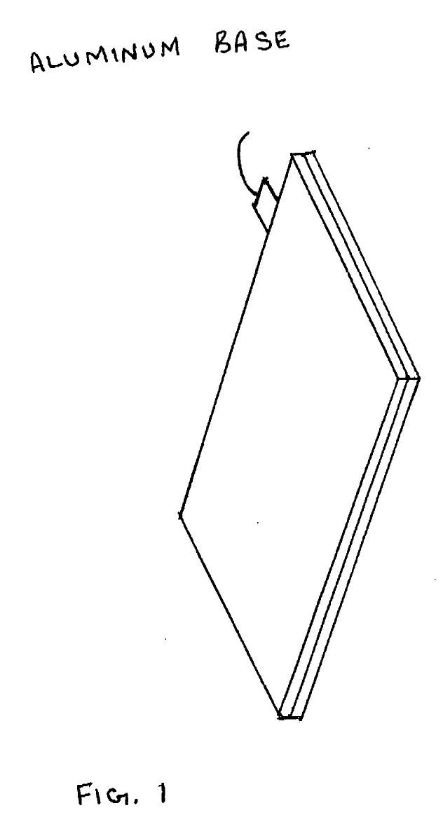

FIG. 2 provides a perspective view of embodiment of Coil or Copper House which is connected to bulb which is used to give induction through hands

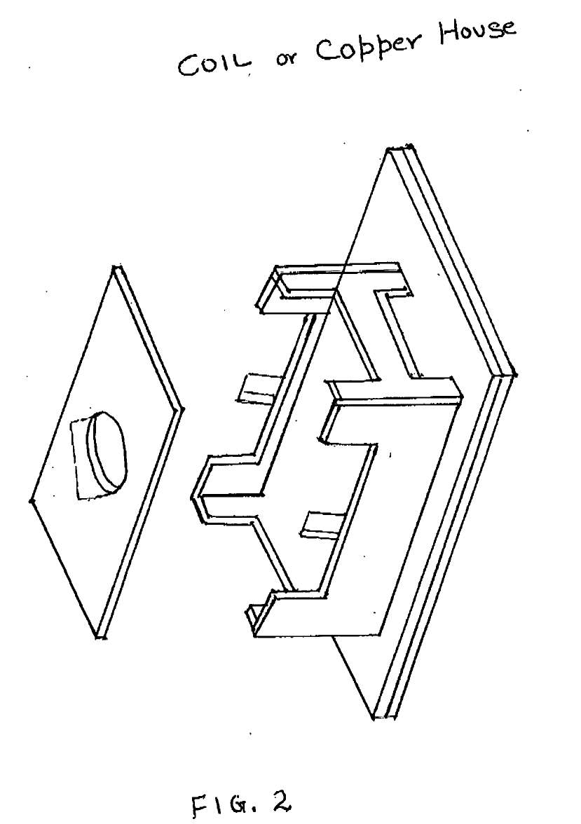

FIG. 3 provides a perspective view of embodiment of Bulb which is connected to Coil or Copper House from where the induction is given to the hands

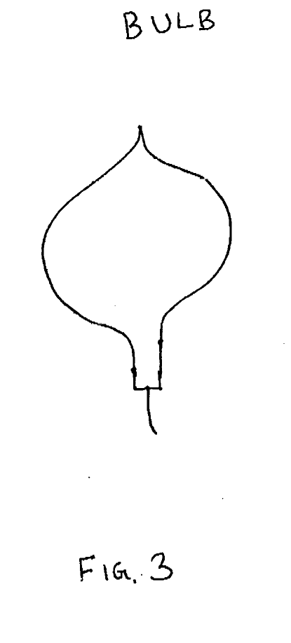

I claim:

1. A whole body integrated Pulsed Electromagnetic Field Therapy (PEMF) system device apparatus comprising:
Aluminum base comprising of glass, aluminum and plastic or Texan or any other such base material; b) Coil or copper house comprising of fan, coil or copper and plastic or any such coil or copper house material; c) glass bulb; and d) electrical unit to give power to: increase cells energy, strengthen immune system and promote cell regeneration.
1) The integrated whole body PEMF system device as claimed in claim 1, when used for 45 minutes to 50 minutes 4 times/day for a specified amount of time, based on the health condition, has helped patients with breast cancer which is on chemotherapy and/or radiation. Patients felt better with PEMF Therapy, headaches, burning, numbness, shooting pains were reduced and had much more energy after chemotherapy. Nausea/vomiting feeling was gone and overall felt much better. All the side effects and complications of chemotherapy and/or radiation were gone, if body responds to the therapy.
2) The integrated whole body PEMF system as claimed in claim 1, when used for 30 minutes to 35 minutes 4 times/day for a specified amount of time, based on the health condition, has helped relieved chronic pain of many patients, if body responds to the therapy.
3) The integrated whole body PEMF system as claimed in claim 1, when used for 30 minutes to 45 minutes 5 times/day for a specified amount of time, based on the health condition, as a low field magnetic stimulation on depression and other psychiatric conditions patients has shown rapid improvement in many psychiatric conditions, if body responds to the therapy.
4) The integrated whole body PEMF system as claimed in claim 1, when used for 30 minutes to 35 minutes 6 times/day for a specified amount of time, based on the health condition, as a low field magnetic stimulation on Alzheimer disease, it appeared PEMF Therapy helped and supported the aging brain through better circulation and improved sleep. Normal circadian rhythm is severely disrupted in AD patients and PEMF's can help improve circadian rhythm which would, in turn, improve sleep, if body responds to the therapy. Patients undergoing therapy showed a relief of their sleep related problems, if body is able to, with no adverse effects from the PEMF Therapy.
5) The integrated whole body PEMF system as claimed in claim 1, when used for 45 minutes to 55 minutes 6 times/day for a specified amount of time, based on the health condition, on Alzheimer disease, it appeared to improve visual memory and enhance visuoconstructive performance. Improvements followed in other cognitive functions such as short term memory, calculations, spatial orientation, judgment and reasoning as well as more level of energy, social interactions increased, and mood appeared to be much better. This demonstrates that some of the mental deficits caused by AD are possibly reversible, if body responds to the therapy and if body is able to and if PEMF Therapy is used on a much longer period of time and other mental deficits even though they are not reversible, but get significant help from PEMF Therapy.
6) The integrated whole body PEMF system as claimed in claim 1, when used for 35 minutes to 40 minutes 3 times/day for a specified amount of time, based on the health condition, has helped patients with diabetes. The patient felt that that the sensations of pain. numbness, itching and tingling subsided after PEMF Therapy. Their glucose levels were little better also, thereby patients felt better. Other complications of diabetes seem to be better also, if body responds to the therapy.
7) The integrated whole body PEMF system as claimed in claim 1, when used for 45 minutes to 50 minutes 5 times/day for a specified amount of time, based on the health condition, has helped patients with heart problems. PEMF's helped lower vascular resistance which in turn tries to, help to make heart function little better. For patients with hypertension or ischemic heart disease, this means improved circulation as well as lipid metabolism, thereby patients feel better, if body responds to the therapy.
8) The integrated whole body PEMF system as claimed in claim 1, when used for 35 minutes to 50 minutes 5 times/day for a specified amount of time, based on the health condition, has helped patients with arthritis. Patients felt that pain owing to arthritis was gone, inflammation and dysfunctions associated with rheumatoid arthritis (RA) and osteoarthritis (OA) was also gone. Patient also said that the use of PEMF for arthritis has shown that it not only reduces the pain in the arthritis condition, but it also affords chondroprotection, exerts anti-inflammatory action and helps in bone remodeling and PEMF therapy has helped significantly in arthritis, if body responds to the therapy and body is able to.
9) The integrated whole body PEMF system as claimed in claim 1, when used for 35 minutes to 45 minutes 5 times/day for a specified amount of time, based on the health condition, has helped patients with ADHD. Patients felt little better paying attention to others, controlling impulsive behavior is better (did not act without thinking about the results), if body responds to the therapy.
10) The integrated whole body PEMF system as claimed in claim 1, when used for 45 minutes to 50 minutes 6 times/day for a specified amount of time, based on the health condition, has helped patients with Lump in the breast (could have been cancer). This might have a normal or abnormal breast change or a benign or not benign (cancer or not cancer) condition. Regardless of the condition, lump disappeared after PEMF Therapy, because body responded to the therapy.
11) The integrated whole body PEMF system as claimed in claim 1, when used for 45 minutes to 50 minutes 5-7 times/day for a specified amount of time, based on the health condition, has helped patients to prevent diseases. New cells are born and old cells die. PEMF used daily usually helps prevent diseases, if body is able to prevent and lets the cycle of life run properly. It takes care of problems as they arise as body's immune system gets stronger; it also prevents health problems from getting worse, if body is able to function properly. PEMF Therapy acts as a cellular defense around the body. It amplifies neuron-transmitters and brain function. It also increases blood oxygen levels and blood circulation. Everyday use helps to balance blood sugar, blood pressure and cholesterol levels. It takes time to see results, and things don't happen overnight. As cellular energy increases, functions of the all the body's different organ systems are given a boost with proper cellular communication. This process helps keep the body healthy and prevents disease, if body is able to do that.
12) The integrated whole body PEMF system as claimed in claim 1, when used for 45 minutes to 50 minutes 6-7 times/day for a specified amount of time, based on the health condition, has helped patients with overall body wellness. Most of other PEMF machines on the market give only a localized effect. These results are usually temporary. PEMF-DS100 therapy supports total body wellness by full body penetration of a pulsed electromagnetic field, PEMF-DS100 therapy is safe, reliable, and effective. It can be used at home if you want to be healthy. Through resonance, PEMF-DS100 therapy stimulates the body and acts to recharge the body's electrical potential and re-energizing the body will help self-healing capabilities of the body, if body is able to do that.
13) The integrated whole body PEMF system as claimed in claim 1, when used for 50 minutes to 55 minutes 6-7 times/day for a specified amount of time, based on the health condition, has helped patients to strengthen their immune system, so that lymphocytes (T cells, B cells and Natural Killer (NK) cells), function properly, if the body is able to. PEMF THERAPY will try to help to improve the functioning ability of all these cells if it's able to.
14) The integrated whole body PEMF system as claimed in claim 1. when used for 45 minutes to 55 minutes 6-7 times/day for a specified amount of time, based on the health condition, has helped patients for better communication between the cells. If the body is able to, it helps to have constant and consistent communication between the cells.
15) The integrated whole body PEMF system as claimed in claim 1, when used for 45 minutes to 55 minutes 6-7 times/day for a specified amount of time, based on the health condition, has helped patients to increase their self healing capabilities which are already in their body, so their cellular functions in regeneration or re-growth are increased, if body is able to re-grow and body responds to the therapy.
16) The integrated whole body PEMF system as claimed in claim 1, when used for 45 minutes to 50 minutes 6-7 times/day for a specified amount of time, based on the health condition, has helped patients to reverse cell injury, if it is possible and body responds to the therapy.
17) The integrated whole body PEMF system as claimed in claim 1, when used for 45 minutes to 55 minutes 6-7 times/day for a specified amount of time, based on the health condition, has helped patients to reduce recovery time needed to overcome infections and illness, if the body is able to.
18) The integrated whole body PEMF system as claimed in claim 1, when used for 45 minutes to 55 minutes 6-7 times/day for a specified amount of time, based on the health condition, has helped patients to increase blood circulation, thereby delivering more oxygen to tissues, which enables your body to remove toxins more efficiently, if the body is able to.
19) The integrated whole body PEMF system as claimed in claim 1, when used for 45 minutes to 55 minutes 5-7 times/day for a specified amount of time, based on the health condition, has helped patients to replenish the body after energy expenditure and also speeds recovery time after surgery or injury, if the body is able to.
20) The integrated whole body PEMF system as claimed in claim 1 when used for 45 minutes to 50 minutes 6-7 times/day for a specified amount of time, based on the health condition, has helped patients to boosts proper and efficient chemical operation within the cells, which increases the body's cellular ability to absorb chemicals (i.e. medications prescribed by doctors or hospital), if body responds to the therapy and is able to.
